(19) United States
(12) Patent Application Publication
(10) Pub. No.: US 2007/0270432 A1
(43) Pub. Date: Nov. 22, 2007

(54) Novel Method

(76) Inventors: James Hagan, Harlow Essex (GB); Neil Upton, Harlow Essex (GB); Andrew Foley, Dublin (IE); Helen Gallagher, Dublin (IE); Ciaran Regan, Dublin (IE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/503,679

(22) PCT Filed: Feb. 4, 2003
(86) PCT No.: PCT/GB03/00462

§ 371 Date: Sep. 12, 2005

(30) Foreign Application Priority Data
Feb. 5, 2002 (GB) 0202680.5
Sep. 30, 2002 (GB) 0222616.5

(51) Int. Cl. A61K 31/495; A61K 31/496; A61P 25/00

(52) U.S. Cl. 514/252.13; 514/255.03

(57) ABSTRACT

The present invention relates to a novel method of promoting neuronal growth within the central nervous system of a mammal and to compounds and pharmaceutical compositions for use in such a method.

This invention relates to a novel method of promoting neuronal growth within the central nervous system of a mammal and to compounds and pharmaceutical compositions for use in such a method.

The widely held belief that the permanent loss of neurons associated with Alzheimer's or Parkinson's disease and injury such as stroke offers no possibility of cellular regeneration has been challenged by the extensive evidence for neural stem cells resident within the adult brain (Gage, F. H. (2000) Science 287, 1433-1438). Neurogenesis and the synaptic plasticity of these newborn cells can be influenced by stress, an enriched environment and physical exercise (van Praag et al., (1999) Proc. Natl. Acad. Sci. USA 96, 13427-13431; Nilsson et al., (1999) J. Neurobiol. 39, 569-578). New cells generated in situ may also be manipulated pharmacologically and integrated into the existing circuitry. Serotonin, via the 5HT1A receptor, or chronic treatment with antidepressants, such as tranylcypromine, reboxetine or fluoxetine, stimulate hippocampal neurogenesis (Gould, E. (1999) Neuropsychopharm. 21, 46S-51S; Malberg et al., (2000) J. Neurosci. 20, 9104-9110; Brezun and Daszuta (2000) 12, 391-396). By contrast, the competitive NMDA receptor antagonist CGP43487 and opiate receptor agonist morphine reduce the rate of hippocampal neurogenesis (Eisch et al., (2000) Proc. Natl. Acad. Sci. USA 97, 7579-7584; Nacher et al., (2001) Eur. J. Neurosci. 13, 512-520).

A convergent set of data suggests the polysialylated form of the neural cell adhesion molecule (PSA-NCAM) to be implicated in the support of structural reorganizations and synaptic plasticity in areas such as the hypothalamus, olfactory bulb, and hippocampus of the adult nervous system (Seki, T. and Arai, Y. (1993) Neurosci. Res. 17, 265-290; Kiss, J. and Rougon, G. (1997) Curr. Opin. Neurobiol. 7, 640-646). Structural plasticity in the adult hippocampus of several mammalian species, including humans, includes the proliferation of neural precursors in the dentate subgranular zone and these newly generated granule neurons transiently express NCAM PSA (Seki, T. and Arai, Y. (1993) J. Neurosci. 13, 2351-2358). Newly generated, polysialylated neurons, presumably arising from the anterior subventricular zone, are also found in associational areas of the cortex, such as the temporal lobe (Doetsch et al. (1997) J. Neurosci. 17, 5046-5061; O'Connell et al., (1997) J. Neurochem. 68, 2538-2546; NíDhuill et al. (1999) J. Neurosci. Res. 55, 99-106; Gould et al. (1999) Science 286, 548-525). Moreover, during the consolidation of either avoidance conditioning or spatial learning paradigms, transient increases in polysialylated cell frequency occur in the 12 h post-training period and are necessary for the accompanying dendritic remodeling observed in rat hippocampus and medial temporal lobe (Fox et al. (1995) J. Neurochem. 65, 2796-2799; Murphy et al. (1996) J. Neurochem. 67, 1268-1274; O'Connell et al. (1997) J. Neurochem. 68, 2538-2546; O'Malley et al. (1998) Neuroscience 87, 607-613; O'Malley et al. (2000) Neuroscience 99, 229-232; Fox et al (2000) J. Neurobiol. 45, 135-141).

Multiple 5-hydroxytryptamine (5-HT) receptors have been identified (5-HT1A/1B/1D/1E/1F, 5-HT2A/2B/2C, 5-HT3A/3B, 5-HT4A/4B, 5-HT5A/5B, 5-HT6 and 5-HT7A/7B/7C/7D) and extensive evidence suggests that 5-HT receptors have a role in learning and memory. A number of antagonists of the 5-HT₆ sub group of 5-HT receptors have been discovered and published in international publication numbers WO 98/27081, WO 98/27058, WO 99/02502, WO 99/37623, WO 99/42465, WO 00/12073, WO 00/12623, WO 01/32646 (all in the name of SmithKline Beecham plc) and these compounds are believed to be of potential use in the treatment of certain CNS disorders such as anxiety, depression, epilepsy, obsessive compulsive disorders, migraine, Alzheimers disease (cognitive memory enhancement), sleep disorders (including disturbances of Circadian Rhythm), feeding disorders such as anorexia and bulimia, panic attacks, withdrawal from drug abuse such as cocaine, ethanol, nicotine and benzodiazepines, schizophrenia, ADHD, disorders associated with spinal trauma and/or head injury such as hydrocephalus and certain GI disorders such as IBS. Relatively high levels of the 5HT6 receptors are found in the molecular layer of the hippocampal dentate gyrus (Gérald et al. (1997) Brain Res. 746, 207-219) where their antagonism may enhance excitability directly or through an intervening inhibitory action on the GABAergic interneurons.

One such compound disclosed as Example 83 in WO 98/27081 is 5-Chloro-3-methylbenzo[b]thiophene-2-sulfonic acid (4-methoxy-3-piperazin-1-ylphenyl)amide hydrochloride, which has also been referred to in the literature as SB-271046. SB-271046 has been characterised as a potent antagonist of human (pKi 8.8-8.9) and rat (pKi 9.0) 5-HT6 receptors. In addition, the compound is over 200-fold selective for 5-HT6 receptors versus 55 other receptors, binding sites and ion channels. SB-271046 is orally bioavailable and increases seizure threshold (an action indicative of anticonvulsant properties) in the rat maximal electroshock seizure threshold test over a wide-dose range (0.1-30 mg/kg) (Routledge et al, (2000) British J. Pharm. 130, 1606-1612). At 10 mg/kg p.o., SB-271046 also produces significant improvements in retention of a spacial memory task in the rat thus highlighting its potential for enhancing cognitive processes in humans (Rogers, D. C. & Hagan, J. J. (2001) Psychopharmacology 158: 114-119.

The inventors of the present invention have found that 5-HT₆ receptor antagonists are capable of increasing basal and learning-induced polysialylated neuron cell frequency in brain regions such as the rat medial temporal lobe and associated hippocampus.

Thus, according to the present invention we provide a method of promoting neuronal growth within the central nervous system of a mammal which comprises the step of administering a 5-HT₆ receptor antagonist.

Preferably, neuronal growth will be promoted within the regions primarily responsible for learning and memory functions, such as the hippocampus or medial temporal lobe regions of the central nervous system of a mammal.

Preferably, the 5-HT₆ receptor antagonist will be administered in the form of a pharmaceutical composition.

Diseases which can be treated by the method of the present invention include neurodegenerative diseases such as Alzheimer's Disease, Parkinson's Disease and stroke.

Wherein said 5-HT₆ receptor antagonist is administered in the form of a pharmaceutical composition it may be prepared in admixture with one or more pharmaceutically acceptable excipients.

As a second aspect of the present invention we provide a use of a 5-HT₆ receptor antagonist in the manufacture of a medicament for promoting neuronal growth within the central nervous system of a mammal.

As a further aspect of the present invention we provide a pharmaceutical composition comprising a 5-HT₆ receptor antagonist for use in promoting neuronal growth within the central nervous system of a mammal.

A pharmaceutical composition of the invention, which may be prepared suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or a pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 200 mg, for example 20 to 40 mg; and such unit doses will preferably be administered once a day, although administration more than once a day may be required; and such therapy may extend for a number of weeks or months.

5-HT₆ receptor antagonists known in the art are of potential use in promoting neuronal growth within the central nervous system of a mammal. For example, those 5-HT₆ receptor antagonists disclosed in International publication numbers WO 98/27081, WO 98/27058, WO 99/02502, WO 99/37623, WO 99/42465, WO 00/12073, WO 00/12623, WO 01/32646 (all in the name of SmithKline Beecham plc) herein incorporated by reference.

In one preferred aspect of the present invention, said 5-HT₆ receptor antagonist is 5-chloro-3-methylbenzo[b]thiophene-2-sulfonic acid (4-methoxy-3-piperazin-1-ylphenyl)amide or a pharmaceutically acceptable salt or solvate thereof, most preferably as the hydrochloride salt.

In a second preferred aspect of the present invention, said 5-HT₆ receptor antagonist is N-(3,5-dichloro-2-methoxy-phenyl)-4-methoxy-3-piperazin-1-yl-benzenesulfonamide or a pharmaceutically acceptable salt or solvate thereof, most preferably as the hydrochloride salt.

The present invention is illustrated by reference to the following Examples:

### EXAMPLES

(a) General Experimental

Experimentally naïve postnatal day 80 (at the time of NCAM-PSA assessment) male Wistar rats were employed in all studies. All animals were housed singly and maintained at 22±2° C. on a standard 12 h-light/dark cycle with food and water available ad libitum. Animals were introduced to the experimental holding rooms at least 3 days prior to the commencement of any study.

Within the Examples, references to SB271046 should be interpreted as references to 5-Chloro-3-methylbenzo[b]thiophene-2-sulfonic acid (4-methoxy-3-piperazin-1-ylphenyl)amide hydrochloride and references to SB399885 should be interpreted as references to N-(3,5-dichloro-2-methoxy-phenyl)-4-methoxy-3-piperazin-1-yl-benzenesulfonamide hydrochloride.

(b) Quantitative Analysis of NCAM PSA Expression

(i) Cryosection Technique

Freshly dissected whole rat brain was carefully coated in optimum cutting temperature (OCT) compound and lowered into a Cryoprep freezing apparatus containing dry-ice-cooled n-hexane. The function of the OCT compound and n-hexane was to provide an even freezing of the tissue, thus avoiding freezing artefacts. Horizontal sections for all studies were cut semi-automatically or automatically on a Microm Series 500 cryostat. Fresh, frozen brain sections (12 μm) were cut at −15° C., while cryoprotected. All sections were prepared on the day of the experiment and were not pre-cut and stored frozen. For the analysis of the NCAM PSA-positive hippocampal dentate granule cell layer/hilus border cells, 10 alternate sections were taken at a level equivalent to −5.6 mm below Bregma (Paxinos and Watson, 1986), at which level this cell population was found to be maximal.

The frequency of polysialylated neurons in the rat medial temporal lobe was also examined following chronic exposure to 5-HT₆ antagonist. These polysialylated neurons, located in layer II of the entorhinal and perirhinal cortex and exhibiting a dorso-ventral increase in frequency, were examined at bregma levels −7.1, −7.6, −8.1 and −8.6.

(ii) Immunohistochemical Protocol

Horizontal cryosections were cut from the frozen tissue at various levels with reference to Bregma (see above), these were thaw-mounted onto glass slides, which were coated with poly-1-lysine diluted 1:1 in dH₂O, and immersion fixed for 30 minutes with 70% ethanol. The sections were then washed twice for 10 minutes each in 0.1M phosphate buffered saline (PBS) and incubated for 20 hours in a humidified chamber at room temperature with the primary antibody diluted 1:500 in PBS containing 1% bovin serum albumin (w/v) and 1% normal goat serum (v/v) to reduce non-specific staining. The humidified chamber prevented the sections from evaporating. The primary antibody was a monoclonal raised against PSA, which was provided by Professor G, Rougon (CNRS UMR 6545, 13288 Marseille, France). On completion of the primary antibody incubation, the sections were washed twice for ten minutes each in PBS and incubated at room temperature for 3 hours in the humidified chamber with the secondary antibody, at a dilution factor of 1:100, again in PBS containing 1% BSA and 1% NGS. The secondary antibody was a goat anti-mouse IgM conjugated to fluorescein (FITC). Following the second incubation, the sections ware again washed twice for ten minutes each in PBS, mounted in the fluorescence enhancing medium Citifluor® and observed for fluorescence with a Leitz DM RB fluorescent microscope.

(iii) Quantitative Evaluation of NCAM PSA Expression

Quantitative image analysis was performed using the Leica Quantimet 500®, a P.C.-based software package, which was connected to the fluorescence microscope with a high sensitivity CCD video camera. Each microscope lens was calibrated for length and area measurements using a 1 mm graticule. The total number of NCAM PSA-immunoreactive neurons on the right dentate granule cell layer/hilar border were counted in 7 alternate 12 μm sections commencing −5.6 mm from Bregma (Paxinos and Watson, 1986), to preclude double counting of the 5-10 μm perikarya. Cell identification was aided by the use of the nuclear counter-stain propidium iodide (40 ng/ml PBS; 60 sec). The number of cells was then divided by the total area of the dentate granule cell layer and multiplied by the average granule cell layer area for a p80 rat, which is 0.15±0.01 mm² at this level. This was done for each section and a mean±SEM was calculated for each brain with the results expressed as PSA-positive cells per unit area. These results were then used to generate the mean±SEM for each animal group. Cell identification was again aided by the use of the nuclear counter-stain propidium iodide (40 ng/ml PBS; 60 sec) with the use of alternate sections eliminating the possibility of double counting. Cell counts were divided by the length of the cortex and multiplied by the average length of the cortex, which was taken to be 10 mm. This was completed for each section and a mean±SEM was calculated for each brain with the results expressed as PSA-positive cells per unit length. These results were used to generate the final mean±SEM for each animal group.

(iv) Water Maze Training

1. Behavioural Assessment

In this protocol animals were introduced to the training environment 5 days prior to training, and individually housed according to standard conditions. Animals were left to habituate to the environment for days 1 and 2 with no handling, on days 3, 4 and 5 animals were handled, their weight monitored and spontaneous behaviour was assessed in open field apparatus for 5 minutes. Open field studies formed an essential part of all training procedures. The open field apparatus consisted of black-painted wood 620 mm long, 620 mm wide, and 150 mm high. The white-painted floor of the apparatus was ruled from side to side, dividing it into a series of boxes 77×77 mm square. Locomotor activity was measured as the number of lines crossed over 300 seconds. Other behaviours assessed were rearing, grooming, piloerection, defecation and posture. These behavioural assessments were invaluable for detecting animals failing to respond to the training schedule or possible unwarranted drug effects that may confound test results.

2. Apparatus

The water maze apparatus consisted of a circular pool (1 m diameter, 80 cm high) specially constructed from established designs in black Perspex. The temperature was maintained at 26° C. by way of a heating element, which was covered by a false bottom with a pump to circulate the water. A platform (11 cm diameter) was submerged 1.5 cm below the water surface, also constructed from black Perspex. During training the platform was hidden in one quadrant of the maze 30 cm from the sidewall. The black Perspex of the maze and platform offer no intramaze cues to guide escape behaviour. However, the training room offers several strong extramaze visual cues to aid the formation of the spatial map necessary for escape learning. An automated tracking system “Water maze 3.1” was employed. This program analyses video images acquired via digital camera and image acquisition board, determining path-length, duration, maximum speed, angle (angle between the initial direction of swim and the endpoint (platform), and the number of entries and duration of swim spent in each quadrant of the water maze.

3. Single Session Water Maze Training

This was the standard paradigm employed to study molecular events associated with learning and memory consolidation, as described previously publications (Murphy et al. (1996) J. Neurochem. 67, 1268-1274). Each trial starts with the rat placed facing the wall of the maze at one of three designated locations. The rat was allowed to explore the maze and the time taken to find the hidden platform within a 60 s criterion period was defined as the escape latency time. On the first trial, rats failing to locate the platform within the 60 s period were placed on it for 10 seconds. In subsequent trials animals failing to locate the platform were not shown it again. Escape latencies were measured over 5 trials with an inter-trial test interval of 300 seconds.

Animals from acute and chronic treatment groups were trained as outlined above. All animals acquired the task as indicated by the decrease in latency to find the platform between trial 1 and trial 5. [Acute study: drug-treated and trained—F (4,19)=3.531; p=0.032; untreated and trained—F (4,19)=7.748; p=0.0014] [Chronic study: drug-treated and trained—F (4,19)=13.345; p<0.0001; untreated and trained—F (4,19)=1.455; p=0.2647] Subsequently, 12 hours following cessation of trial 5 the animals were sacrificed by cervical dislocation, brain tissue dissected free and cryopreserved for quantification of NCAM polysialylation as above.

(v) Data Analysis

NCAM PSA-positive cell numbers were obtained from each animal group. Results were expressed as mean±SEM with at least 3-6 values per group and analysed by ANOVA or unpaired non-parametric, Student's t-test, as indicated.

(c) Quantitative Analysis of Bromodeoxyuridine (BrdU) Expression

(i) Tissue Preparation

Following transcardial perfusion with a 4% paraformaldehyde solution at pH 7.4, brains are removed and stored in the same fixative overnight. Subsequently, the brains are carefully coated in optimum cutting temperature (OCT) compound and lowered into a Cryoprep freezing apparatus containing dry-ice-cooled n-hexane. The function of the OCT compound and n-hexane is to provide an even freezing of the tissue, thus avoiding freezing artifacts.

(ii) Cryosection Technique

Sections for all studies are cut manually on a Microm Series 500 cryostat and are horizontal in orientation. Fresh, frozen brain sections (50 μm) are cut at -25° C., while cryoprotected. All sections are prepared on the day of the experiment and are not pre-cut and stored frozen. This provides for optimal tissue morphology. For the analysis of the BrdU-immunopositive hippocampal dentate granule cell layer cells, 8 free-floating sections are obtained from each brain and stored in cryoprotectant (0.32M Sucrose). These are taken at 50 0μm intervals commencing at a level equivalent to −4.1 mm below Bregma.

(iii) Immunohistochemical Protocol

The sections are transferred from cryoprotectant and washed three times for 5 minutes each in a 0.1M PBS solution containing 5 mM MgCl₂ and 1 mM CaCl₂ (required for the stability of DNAse enzyme). For DNA denaturisation the sections are incubated at 37° C. for 1 hour in DNAse (1000 units/ml). The sections are again washed and blocked with 10% w/v NGS for 30 minutes, then incubated for 20 hours at room temperature with the primary antibody (anti-BrdU rat IgG, Harlan UK), diluted 1:100 in PBS containing 10% NGS (v/v) to reduce non-specific staining. Subsequently, the sections are washed and incubated at room temperature for 1 hour with the secondary antibody (Alexa 488-conjugated goat anti-rat IgG, Molecular Probes UK), diluted 1:200, again in PBS containing 10% NGS. Following the second incubation, the sections are again washed and mounted in Citifluor.

(iv) Quantitative Evaluation of BrdU Expression

The frequency of BrdU-immunoreactive cells in the right dentate granule cell layer is counted in 10 random sections throughout the hippocampus. Then quantitative image analysis is performed using Leica Quantimet 500 software, to determine the area of the granule cell layer in each section and then the granule cell layer volume by the Cavalieri method. The total number of BrdU-immunopositive cells per granule cell layer is then established from the resultant cell density and granule cell layer volume, and is used to generate the mean±SEM number of BrdU-immunopositive cells per granule cell layer for each animal group. Statistical analysis employs the Student's t-test.

### Example 1

### Effect of Chronic Administration of SB271046 Upon Neuronal Cell Growth Within the Hippocampus

Postnatal day 40 male animals (maintained in accordance with the general procedure detailed in section (a) above) were administered 3, 10 or 20 mg/kg SB271046 for 40 days by gavage. Drug administration ceased 24 h prior to animal sacrifice. Animal weight gain and general physical condition was monitored daily. Methylcellulose (1% w/v) treated controls and use of the antipyschotic clozapine were employed for comparison. NCAM PSA expression was then quantified for each of the 5 groups of animals (eg. control, 3, 10 and 20 mg/kg SB271046 and clozapine) in accordance with the general procedure detailed in sections (b)(i)-(iii) above.

The resultant data obtained was analysed as described in section (b)(v) above and SB271046 was found to significantly increase the frequency of polysialylated neurons in the subventricular zone of the rat hippocampal dentate gyrus, in a dose-dependent manner, as detailed in Table 1 below. This effect was not observed in the vehicle-treated control or with the antipsychotic clozapine. These polysialylated neurons are represented by fluorescent cells located at the granule cell layer/hilar border and their dendrites extend into the molecular layer of the hippocampal dentate gyrus.

TABLE 1

| | PSA immunopositive |
|---|---|
| Treatment | cell frequency |
| control | 63.4 ± 3.5 |
| SB271046 (3 mg/kg) | 70.3 ± 3.9 |
| SB271046 (10 mg/kg) | 82.4 ± 1.7* |
| SB271046 (20 mg/kg) | 85.8 ± 8.4* |
| clozapine (5 mg/kg) | 69.8 ± 1.6 |

*P < 0.05 versus control, one-way ANOVA; n = 6 in all cases.

The frequency of polysialylated neurons in the rat medial temporal lobe was also increased following chronic exposure to SB271046 (20 mg/kg), as detailed in Table 2 below. These polysialylated neurons are located in layer II of the entorhinal and perirhinal cortex and exhibit a dorso-ventral increase in frequency. At bregma levels −7.1, −7.6 and −8.6 polysialylated cell frequency was significantly increased as compared to the methylcellulose-treated control animals. No significant increase in polysialylated cell frequency was observed at bregma level −8.1.

TABLE 2

PSA immunopositive cell frequency

| | | SB271046 |
|---|---|---|
| Bregma level (mm) | Control | (20 mg/kg)-treated |
| −7.1 | 47.3 ± 4.2 | 61.7 ± 1.7* |
| −7.6 | 52.6 ± 3.8 | 69.9 ± 0.9* |
| −8.1 | 111.1 ± 6.9 | 125.4 ± 3.5 |
| −8.6 | 141.3 ± 4.9 | 178.3 ± 12.2* |

Control group is significantly different to treated group by two-way ANOVA. Significant differences between each bregma level is indicated by an asterisk (p<0.05, unpaired, non-parametric Student's t-test), n=3 in all cases.

### Example 2

### Effect of Acute and Chronic Administration of SB271046 Upon Learning Induced Activation of Neuronal Cell Growth Within the Hippocampus

Postnatal day 80 male animals (maintained in accordance with the general procedure detailed in section (a) above) were administered 20 mg/kg SB271046 by gavage 30 min before water maze training in accordance with the protocol described in section (b)(iv) above (acute administration) or postnatal day 40 male animals were administered 20 mg/kg SB271046 for 40 days by gavage and water maze trained in accordance with the protocol described in section (b)(iv) above on postnatal day 80 (chronic administration). Methylcellulose (1% w/v) treated controls were employed for comparison. NCAM PSA expression was then quantified for each of the 4 groups of animals (eg. untrained and trained controls and animals administered with 20 mg/kg SB271046) in accordance with the general procedure detailed in sections (b)(i)-(iii) above.

The resultant data obtained was analysed as described in section (b)(v) above and acute administration of SB271046 was found to significantly increase the frequency of polysialylated neurons in the subventricular zone of the rat hippocampal dentate gyrus at 12 h following water maze training as compared to untrained animals receiving the drug and, also, in respect of the trained but drug-naive animals (Table 3). Similar results were obtained with animals chronically exposed to SB271046 (Table 4).

TABLE 3

| | | PSA immunopositive |
|---|---|---|
| Treatment | | cell frequency |
| 1. | Untrained control | 65.2 ± 2.4 |
| 2. | 12 h post-training control | 85.3 ± 1.8 |
| 3. | SB271046 (20 mg/kg)-treated | 64.2 ± 4.3 |
| | untrained control | |
| 4. | SB271046 (20 mg/kg)-treated | 96.0 ± 5.5 |
| | 12 h post-training | |

Statistical evaluation of Table 3

| Data points compared | p value |
|---|---|
| 1 versus 2 | 0.0003 |
| 3 versus 4 | 0.0044 |
| 2 versus 4 | 0.0361 |

Unpaired non-parametric, Student's t-test, n = 3-6 in all cases.

TABLE 4

| | | PSA immunopositive |
|---|---|---|
| Treatment | | cell frequency |
| 1. | Untrained control | 63.4 ± 3.5 |
| 2. | 12 h post-training control | 81.1 ± 1.6 |
| 3. | SB271046 (20 mg/kg)-treated | 85.8 ± 1.3 |
| | untrained control | |
| 4. | SB271046 (20 mg/kg)-treated | 109.8 ± 1.8 |
| | 12 h post-training | |

Statistical evaluation of Table 4

| Data points compared | p value |
|---|---|
| 1 versus 2 | 0.009 |
| 3 versus 4 | 0.0484 |
| 2 versus 4 | 0.0002 |

Unpaired non-parametric, Student's t-test, n = 3 in all cases.

### Example 3

### Effect of Chronic Administration of SB399885 Upon Neuronal Cell Growth Within the Hippocampus

Postnatal day 40 male animals (maintained in accordance with the general procedure detailed in section (a) above) were administered 3, 10 or 20 mg/kg SB399885 for 40 days by gavage. Drug administration ceased 24 h prior to animal sacrifice. Animal weight gain and general physical condition was monitored daily. Methylcellulose (1% w/v) treated controls were employed for comparison. An additional SB271046 (20 mg/kg) treatment group was utilised to ensure comparable results with previous studies. NCAM PSA expression was then quantified in accordance with the general procedure detailed in sections (b)(i)-(iii) above.

The resultant data obtained was analysed as described in section (b)(v) above and SB399885 was found to significantly increase the frequency of polysialylated neurons in the subventricular zone of the rat hippocampal dentate gyrus, in a dose-dependent manner, as detailed in Table 5 below. This effect was not observed in the vehicle-treated control. These polysialylated neurons are represented by fluorescent cells located at the granule cell layer/hilar border and their dendrites extend into the molecular layer of the hippocampal dentate gyrus.

TABLE 5

| | PSA immunopositive |
|---|---|
| Treatment | cell frequency |
| control | 58.7 ± 3.9 |
| SB399885 (3 mg/kg) | 81.4 ± 5.3* |
| SB399885 (10 mg/kg) | 87.4 ± 5.4* |
| SB399885 (20 mg/kg) | 104.2 ± 4.4* |
| SB271046 repeat (20 mg/kg) | 78.3 ± 3.4* |

*P < 0.05 versus control, Student's t-test; n = 6 in all cases. SB399885 versus control, one-way ANOVA, F(3, 23) = 15.3; P < 0.0001

Moreover, analysis of variance shows the dose dependent increase in basal frequency of hippocampal polysialylated neurons following chronic SB399885 treatment was significantly greater than that observed following chronic SB271046 administration in Example 1 above (F[1,21]=5.882; P=0.0244). Furthermore, in this experiment, there was no difference in the frequency of polysialylated neurons in the subventricular zone of the rat hippocampal dentate gyrus in SB271046-treated animals as compared to that observed in Example. 1

### Example 4

### Effect of Chronic Administration of SB399885 on Hippocampal Polysialylated Neuron Cell Frequency Following Water Maze Training

Postnatal day 40 male animals (maintained in accordance with the general procedure detailed in section (a) above) were administered 20 mg/kg SB399885 for 40 days by gavage and water maze trained in accordance with the protocol described in section (b)(iv) above on postnatal day 80 (chronic administration). Methylcellulose (1% w/v) treated controls were employed for comparison. NCAM PSA expression was then quantified in accordance with the general procedure detailed in sections (b)(i)-(iii) above.

The resultant data obtained was analysed as described in section (b)(v) above and chronic administration of SB399885 was found to significantly increase the frequency of polysialylated neurons in the subventricular zone of the rat hippocampal dentate gyrus at 12 h following water maze training as compared to untrained animals receiving the drug and, also, in respect of the trained but drug-naive animals (Table 6).

TABLE 6

| | | PSA immunopositive |
|---|---|---|
| Treatment | | cell frequency |
| 1. | Untrained control | 58.7 ± 3.9 |
| 2. | 12 h post-training control | 91.3 ± 6.5 |
| 3. | SB399885 (20 mg/kg)-treated | 104.2 ± 4.4 |
| | untrained control | |
| 4. | SB399885 (20 mg/kg)-treated | 125.9 ± 4.7 |
| | 12 h post-training | |

Statistical evaluation

| Data points compared | p value |
|---|---|
| 1 versus 2 | 0.0027 |
| 3 versus 4 | 0.0189 |
| 2 versus 4 | 0.0127 |

Unpaired non-parametric, Student's t-test, n = 3 in all cases.

Moreover, the significant increase in the observed frequency of hippocampal polysialylation neurons 12 h following water maze training in those animals chronically administered SB399885 (20 mg/kg), was significantly greater than that observed following SB271046 treatment in Example 2 (Student's t-test; P=0.0337).

### Example 5

### Effect of Chronic Administration of SB271046 or SB399885 on Hippocampal Neurogenesis

Postnatal day 40 male animals (maintained in accordance with the general procedure detailed in section (a) above) were administered 20 mg/kg SB271046 or SB399885 for 40 days by gavage (chronic administration). For the last eight days of the study animals from each treatment group are administered bromodeoxyuridine (BrdU, 100 mg/kg, i.p.), which is a marker of DNA synthesis that has been used extensively to study brain neurogenesis (Gage (2002) J. Neurosci. 22, 612-613). Drug administration ceased 24 h prior to animal sacrifice. Animal weight gain and general physical condition was monitored daily. Methylcellulose (1% w/v) treated controls were employed for comparison. BrdU expression was then quantified in accordance with the general procedure detailed in section (c) above.

Neither SB271046 nor SB399885 chronic administration significantly altered the expression of BrdU-immunopositive cells in the hippocampal dentate granule cell layer as compared to vehicle-treated controls, as detailed in Table 7 below, demonstrating the ability of both compounds to activate NCAM PSA expression without altering neurogenic rate.

TABLE 7

| | BrdU-immunopositive cell |
|---|---|
| Treatment | number/granule cell layer |
| control | 2432 ± 435.8 |
| SB271046 (20 mg/kg) | 2332 ± 136.5 |
| control | 2456.7 ± 250.9 |
| SB399885 (20 mg/kg) | 2296.7 ± 49.1 |

The patents and patent applications described in this application are herein incorporated by reference.

1. A method of promoting neuronal growth within the central nervous system of a mammal which comprises the step of administering a 5-HT₆ receptor antagonist.

2. A method according to claim 1 wherein said 5-HT₆ receptor antagonist is administered in the form of a pharmaceutical composition.

3. A method according to claim 1 wherein said 5-HT₆ receptor antagonist is 5-chloro-3-methylbenzo[b]thiophene-2-sulfonic acid (4-methoxy-3-piperazin-1-ylphenyl)amide or a pharmaceutically acceptable salt or solvate thereof.

4. A method according to claim 3 wherein the pharmaceutically acceptable salt is the hydrochloride.

5. A method according to claim 1 wherein said 5-HT₆ receptor antagonist is N-(3,5-dichloro-2-methoxy-phenyl)-4-methoxy-3-piperazin-1-yl-benzenesulfonamide or a pharmaceutically acceptable salt or solvate thereof.

6. A method according to claim 5 wherein the pharmaceutically acceptable salt is the hydrochloride.

7-17. (canceled)
